(19) United States
(12) Patent Application Publication
(10) Pub. No.: US 2023/0034970 A1
(43) Pub. Date: Feb. 2, 2023

(54) FILTER-BASED ARRHYTHMIA DETECTION

(71) Applicant: Medtronic, Inc., Minneapolis, MN (US)

(72) Inventors: Ya-Jian Cheng, Lino Lakes, MN (US); Eduardo N. Warman, Maple Grove, MN (US); Jeffrey M. Gillberg, Coon Rapids, MN (US); Abhijit Kadrolkar, St. Paul, MN (US); Shantanu Sarkar, Roseville, MN (US); Kevin T. Ousdigian, Shoreview, MN (US)

(21) Appl. No.: 17/387,728

(22) Filed: Jul. 28, 2021

(51) Int. Cl. A61B 5/0245; A61B 5/00

(52) U.S. Cl. CPC A61B 5/0245; A61B 5/7264; A61B 5/7225; A61B 5/6869; A61B 2560/0223

(57) ABSTRACT

This disclosure is directed to a medical system and technique for a filter-based approach to arrhythmia detection. In one example, the medical system comprises one or more sensors configured to sense physiological parameter(s); sensing circuitry configured to generate patient data based on the sensed physiological parameter(s), the patient data comprising signal data to represent cardiac activity of the patient; and processing circuitry configured to: detect a cardiac arrhythmia for the patient based on a classification of the signal data in accordance with a machine learning model, wherein the machine learning model comprises filter(s) for at least one portion of the signal data, wherein the at least one filter corresponds to a feature set that maps to the cardiac activity represented by the portion(s) of the signal data; and generate for display output data indicative of a positive detection of the cardiac arrhythmia.

### FIELD

The disclosure relates generally to medical systems and, more particularly, medical systems configured to analyze cardiac signals.

### BACKGROUND

Medical systems may monitor various data (e.g., an electrocardiogram (ECG) or a cardiac electrogram (EGM)) of a patient or a group of patients to detect changes in health. In some examples, the medical system may monitor the cardiac EGM to detect one or more types of arrhythmia, such as bradycardia, tachycardia, fibrillation, or asystole (e.g., caused by sinus pause or Atrioventricular block (AV block)). In some examples, the medical system may include one or more of an implantable medical device or a wearable device to collect various measurements used to detect changes in patient health. In some examples, medical systems may include one or more devices configured to deliver therapy to treat conditions. The delivery of therapy may be based on the monitored data.

### SUMMARY

A cardiac EGM may include signal data (e.g., one-dimensional signal data) representing electrical activity of the heart of a patient. The signal data may encode information useful in detecting changes to the patient's cardiac health, and therefore, conventional medical systems employ various mechanisms to analyze the cardiac EGM for indicia of some malady such as a cardiac arrhythmia. However, to help account for physiological differences between different patients (e.g., even patients with similarities in cardiac physiology and/or treatment/therapy delivery), medical systems such as those described herein employ (e.g., one-dimensional) filters that are tailored to the patient's cardiac activity (e.g., morphology of specific wavelets). These filters may be referred to as mission-critical filters or personalized filters; each filter, regardless of characterization in the present disclosure, may encode non-random pattern information derived from a specific portion (e.g., decomposition layer) of a (training) set of cardiac EGM segments indicative of one or more cardiac arrhythmias.

In general, the present disclosure is directed to medical systems, devices, and techniques that potentially benefit patients by identifying cardiac arrhythmias from sensor data describing a given patient's physiological parameters. The techniques include applying a machine learning model to the cardiac EGM in order to determine whether the cardiac EGM is evidence of one or more cardiac arrhythmias.

In one example, a medical system comprises: one or more sensors configured to sense cardiac activity of a patient; sensing circuitry configured to generate signal data to represent the cardiac activity of the patient; and processing circuitry configured to: detect a cardiac arrhythmia for the patient based on a classification of the cardiac activity in accordance with a machine learning model, wherein the machine learning model comprises at least one filter corresponding to a feature set of the patient and configured for application to at least one portion of the signal data; and generate for display output data indicative of a positive detection of the cardiac arrhythmia.

In another example, a method comprises: generating, by sensing circuitry coupled to one or more sensors, signal data to represent cardiac activity of the patient; detecting, by processing circuitry, a cardiac arrhythmia for the patient based on a classification of the signal data in accordance with a machine learning model, wherein the machine learning model comprises at least one filter that is configured for application to at least one portion of the signal data and maps to a feature set indicative of a cardiac physiology of the patient; and generating, by the processing circuitry, output data indicative of a positive detection of the cardiac arrhythmia.

In another example, a non-transitory computer-readable storage medium comprises program instructions that, when executed by processing circuitry of a medical system, cause the processing circuitry to: generate patient data corresponding to at least one physiological parameter of the patient, wherein the patient data comprises signal data to represent electronic activity of a heart of the patient, wherein the medical system comprises one or more sensors configured to sense the electrical activity and sensing circuitry, coupled to the one or more sensors, configured to generate the signal data; detect a cardiac arrhythmia for the patient based on a classification of the patient data in accordance with a machine learning model configured for the at least one physiological parameter of the patient, wherein the machine learning model comprises a plurality of filters of which at least one filter is applied, based on the patient data, to at least one portion of the signal data; and generate output data indicative of a positive detection of the cardiac arrhythmia.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example medical system in conjunction with a patient.

FIG. 2 is a functional block diagram illustrating an example configuration of the implantable medical device (IMD) of the medical system of FIG. 1.

FIG. 3 is a conceptual side-view diagram illustrating an example configuration of the IMD of FIGS. 1 and 2.

FIG. 4 is a functional block diagram illustrating an example configuration of the external device of FIG. 1.

FIG. 5 is a block diagram illustrating an example system that includes an access point, a network, external computing devices, such as a server, and one or more other computing devices, which may be coupled to the IMD and external device of FIGS. 1-4.

FIG. 6 is a flow diagram illustrating an example operation for a filter-based approach to detecting a cardiac arrhythmia.

FIG. 7 is a flow diagram illustrating an example operation for generating filters that are derived from on a decomposition of at least one cardiac EGM of one or more patients.

Like reference characters denote like elements throughout the description and figures.

### DETAILED DESCRIPTION

A variety of types of medical devices sense cardiac activity. Some medical devices that sense cardiac EGMs are non-invasive by, for example using a plurality of electrodes placed in contact with external portions of the patient, such as at various locations on the skin of the patient. The electrodes used to monitor the cardiac EGM in these non-invasive processes may be attached to the patient using an adhesive, strap, belt, or vest, as examples, and electrically coupled to a monitoring device, such as an electrocardiograph, Holter monitor, or other electronic device. The electrodes are configured to sense electrical signals associated with the electrical activity of the heart or other cardiac tissue of the patient, and to provide these sensed electrical signals to the electronic device for further processing and/or display of the electrical signals. The non-invasive devices and methods may be utilized on a temporary basis, for example to monitor a patient during a clinical visit, such as during a doctor's appointment, or for example for a predetermined period of time, for example for one day (twenty-four hours), or for a period of several days.

External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities, and may periodically transmit collected data to a network service, such as the Medtronic Carelink™ Network.

Implantable medical devices (IMDs) may also sense and monitor cardiac EGMs. The electrodes used by IMDs to sense cardiac EGMs are typically integrated with a housing of the IMD and/or coupled to the IMD via one or more elongated leads. Example IMDs that monitor cardiac EGMs include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic plc. Some IMDs that do not provide therapy, e.g., implantable patient monitors, sense cardiac EGMs. One example of such an IMD is the Reveal LINQ™ Insertable Cardiac Monitor, available from Medtronic plc, which may be inserted subcutaneously. Such IMDs may facilitate relatively longer-term monitoring of patients during normal daily activities, and may periodically transmit collected data to a network service, such as the Medtronic Carelink™ Network.

Regardless of which type or types of devices are used, there are number of factors affecting device performance. A noise signal, which may be referred to as an artifact, may appear in a sensed cardiac EGM and the presence of the noise signal in the sensed cardiac EGM may cause circuitry for detecting depolarizations, e.g., R-waves, to wrongly detect the noise signal as a depolarization. These types of improper sensing of depolarizations may lead to improper analysis of the actual cardiac activity occurring with respect to the patient being monitored. Given that a number of devices employ machine learning models, inaccurate and/or noisy data may misrepresent the cardiac activity of the patient and cause a model to make a false determination. For example, these types of improper sensing of depolarizations may potentially trigger a false-positive indication of a cardiac event, such as asystole, that is not actually occurring in the patient. Such false-positive indications could lead to incorrect assessment of the patient condition, including provision of therapy and/or sending false alerts to medical personnel responsible for the care of the patient being monitored. Low pass filtering of the cardiac EGM generally does not help solve these problems because these types of noise signals and amplitude variations may occur at frequencies near or below that of the cardiac signals.

Medical systems according to this disclosure implement techniques that a medical device, such as those described above, may employ when analyzing the cardiac activity of a patient. These techniques introduce a filter-based approach to determining whether a sensed cardiac EGM of the patient is indicative of a cardiac event (e.g., an arrhythmia). Under the filter-based approach, the device is able to provide the patient with improved and personalized medical care. In some instances, the device achieves a reduction in false determinations while device components require less in resource capacities for normal device operation.

Conventional approaches prescribe random filters, and devices implementing conventional approaches may be easily adapted to implement the filter-based approach and realize its benefits by replacing one or more random filters with personalized/calibrated filters that better fit a morphology of signal data for the sensed cardiac EGM.

Instead of using a random filter or a generic filter, the present disclosure introduces personalized and calibrated filters that provide a number of potential benefits and advantages to patient medical devices. In particular, there are additional benefits and advantages to having one-dimensional personalized/calibrated filters. For example, when incorporated in a machine learning model, the one-dimensional personalized/calibrated filters consume fewer resources (e.g., fewer neurons) for each application. When compared to random filters and multi-dimensional filters (e.g., kernels), fewer training samples are utilized for training the one-dimensional personalized/calibrated filters.

The present disclosure describes a number of techniques to generate personalized/calibrated filters. Some example filters may be derived from a decomposition of the sensed cardiac EGM into principal components, wavelets, and/or any other decomposition scheme. Other example filters may be pre-determined/trained to accurately identify wavelets and/or principal components based on expected cardiac activity for the patient or similar patients. Yet another filter may be pre-determined/trained to detect one or more types of cardiac arrhythmias based on the patient's cardiac physiology. In this manner, the techniques of this disclosure may advantageously enable improved accuracy in the identification of true cardiac episodes and, consequently, better evaluation of the condition of the patient.

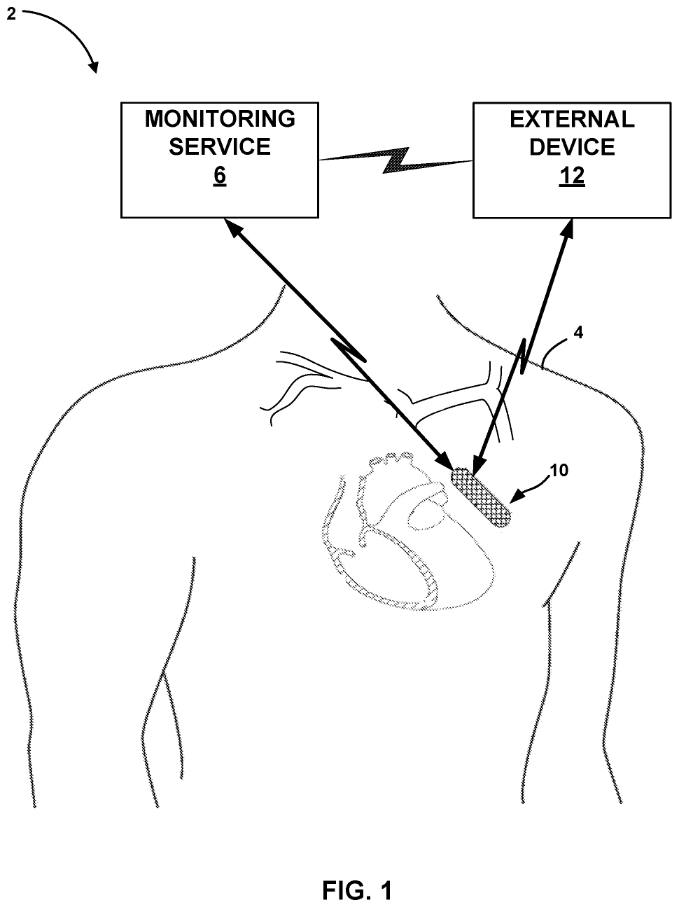

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the LINQ™ ICM.

As described herein, monitoring service 6 is configured to connect with IMD 10 via a wireless communication link and (e.g., automatically) such that IMD 10 is operative to accurate determine whether patient 4's cardiac activity is indicative of a cardiac episode; in such a case, IMD 10 may not be applicable to other patients, especially those unlike patient 4 with respect to personal cardiac activity.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, personal digital assistant, or another computing device that may run an application that enables the computing device to interact with IMD 10.

External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

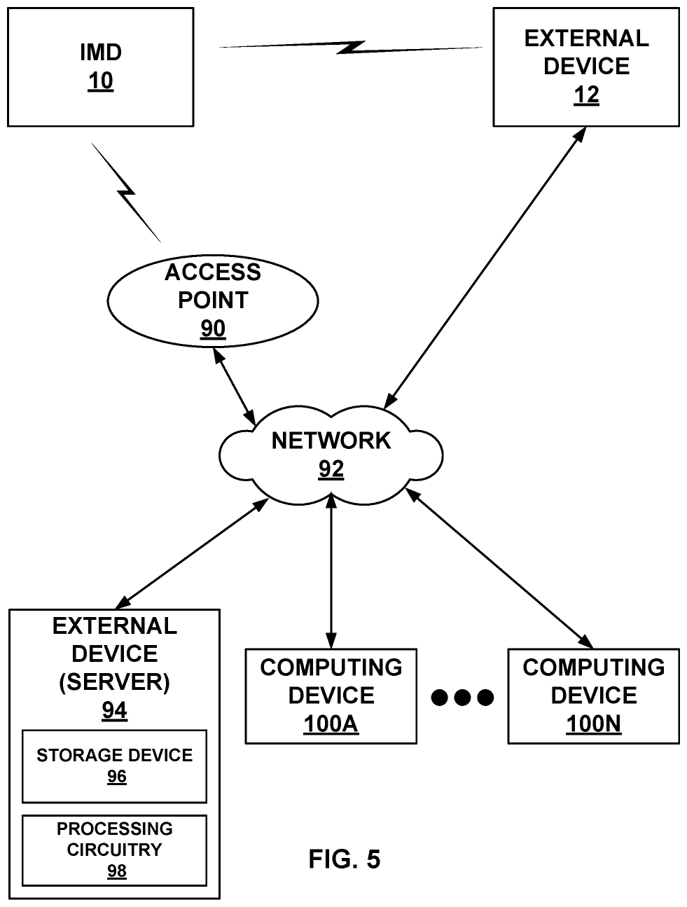

External device 12 may be used to configure operational parameters for IMD 10. External device 12 may be used to retrieve data from IMD 10. The retrieved data may include values of physiological parameters measured by IMD 10, indications of episodes of arrhythmia or other maladies detected by IMD 10, and physiological signals recorded by IMD 10. For example, external device 12 may retrieve cardiac EGM segments recorded by IMD 10 due to IMD 10 determining that an episode of asystole or another malady occurred during the segment. As will be discussed in greater detail below with respect to FIG. 5, one or more remote computing devices may interact with IMD 10 in a manner similar to external device 12, e.g., to program IMD 10 and/or retrieve data from IMD 10, via a network.

Processing circuitry of medical system 2, e.g., of IMD 10, external device 12, and/or of one or more other computing devices, may be configured to perform the example techniques for monitoring cardiac activity of patient 4 for cardiac events including arrhythmias and other types of cardiac episodes. The cardiac activity may be represented by signal data, and in some examples, the signal data may refer to electrical activity of a heart of patient 4. A decomposition of the signal data may refer to a partition of the cardiac activity into portions (e.g., decomposition layers) of which each portion includes wavelet data, principal component data, and/or other data as described herein. The signal data may include a one-dimensional vector representing (e.g., one or more samples of) a cardiac EGM (e.g., signal), and that cardiac EGM may include a number of decomposition layers where each layer encodes informational attributes (e.g., a morphology, a timing, and an amplitude) of a portion of the cardiac activity of patient 4. Processing circuitry of medical system 2 may determine pattern information for a particular wavelet (e.g., R-wave or P-wave) based on at least one example layer including that particular wavelet. That pattern information may represent R-waves or P-waves and their specific morphology in the cardiac activity for patient 4. Processing circuitry of medical system 2 may use the pattern information to generate a filter to identify R-waves or P-waves in the signal data of patient 4. Instead of a random filter or a generic filter, processing circuitry of medical system 2 may employ the above filter to analyze the cardiac activity of the R-waves or P-waves of patient 4 for indicia of the cardiac arrhythmia.

In some examples, the processing circuitry of medical system 2 analyzes the signal data (e.g., a cardiac EGM sensed by IMD 10) with a filter-based approach in which at least one filter is derived. In general, the techniques of the present disclosure demonstrate how to configure a filter to be effective in detecting cardiac arrhythmias in the recorded cardiac activity of patient 4. In one example, the processing circuitry of medical system 2 generates an example filter to encode pattern information for one or more portions of the signal data. The pattern information may define a morphology of a particular wavelet, a principal component, and/or another decomposition layer of the signal data.

Although described in the context of examples in which IMD 10 that senses the cardiac EGM comprises an insertable cardiac monitor, example systems including one or more implantable or external devices of any type configured to sense a cardiac EGM may be configured to implement the techniques of this disclosure.

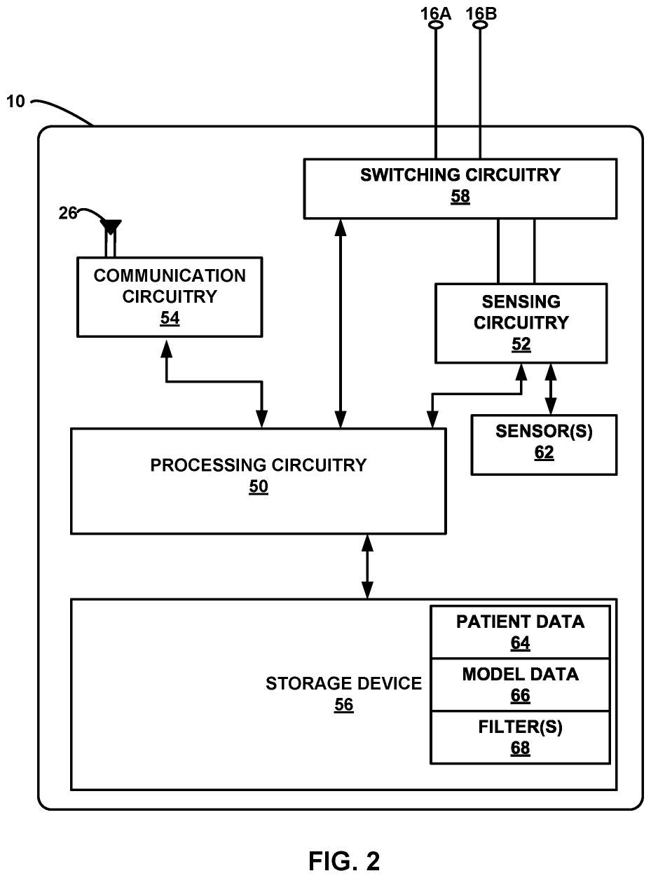

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense a cardiac EGM, as controlled by processing circuitry 50. Sensing circuitry 52 may sense signals from electrodes 16, e.g., to produce a cardiac EGM, in order to facilitate monitoring the electrical activity of the heart. Sensing circuitry 52 also may monitor signals from sensors 62, which may include one or more accelerometers, pulse oximeters, pressure sensors, and/or optical sensors, as examples. In some examples, sensing circuitry 52 may include one or more filters and amplifiers for filtering and amplifying signals received from electrodes 16 and/or sensors 62. Sensing circuitry 52 may further include a rectifier, a comparator, and/or an analog-to-digital converter, in some examples.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include patient data 64, model data 66, and/or filter(s) 68.

Sensing circuitry 52 may be configured to generate patient data 64 based on sensed physiological parameter(s). In general, electrodes 16, sensors 62, and/or other sensors are configured to sense physiological parameter(s) corresponding to cardiac physiology of patient 4 and then, via the above signals, transmit the sensed physiological parameter(s). As such, patient data 64 includes signal data to represent cardiac activity of patient 4.

Sensing circuitry 52 may provide one or more digitized cardiac EGM signals as signal data to processing circuitry 50 for a determination to whether the signal data includes sufficient evidence for a cardiac arrhythmia according to the techniques of this disclosure. Model data 66 may define a machine learning model that processing circuitry 50 may apply to the signal data to facilitate the determination. Processing circuitry 50 may detect the cardiac arrhythmia for patient 4 based on a classification of the signal data in accordance with the machine learning model. Processing circuitry 50 may use the machine learning model to compute a likelihood probability for the arrhythmia and if that likelihood probability exceeds a threshold, a positive detection of the cardiac arrhythmia may be the most probable classification of the signal data.

Model data 66 may define the machine learning model (e.g., a neural network, a probability distribution, a mathematical function, and/or the like) to include one or more filters 68 as part of model prediction logic. The machine learning model may include, for each of a plurality of decomposition layers, a set of one or more filters derived from data associated with that respective one of the plurality of decomposition layers. Model data 66 may prescribe a number of purposes for the one or more filtered datasets. First example filter 68 may be configured to generate a filtered dataset to be part of model input. Processing circuitry 50, when applying first example filter 68 to signal data (e.g., a cardiac EGM), modifies at least one of an amplitude, a timing, or a morphology of principal component data or wavelet data of the at least a portion of the signal data (e.g., at least one decomposition layer of the cardiac EGM).

A decomposition layer generally refers to a portion of the signal data (e.g., window of cardiac EGM) and a type of the decomposition layer of interest corresponds to same or substantially similar cardiac activity. Examples of decomposition layer of interest include, but are not limited to, R-waves, P-waves, QRS-waves, T-waves, flutter waves, VT-waves, AT-waves, QT sections, PR sections, and combinations of above. These examples may be further decomposed (e.g., into sub-layers) by feature sets. In this manner, P-waves for patients having a same feature set (e.g., disease group and device group) and cardiac physiology may be used to derive a calibrated filter for filter(s) 68 that is more efficient and accurate than other filters. The calibrated filter may be referred to as a P-wave hunting filter and, for these patients, configured to be effective (e.g., most effective) when used for identifying P-waves in their cardiac EGMs. If the P-wave hunting filter is calibrated for the patients sharing the same device group, the P-wave hunting filter accounts for non-trivial differences between device groups, such as when a P-wave location is based on device marker channel. If the patients share the same type of device, the P wave location is based on a marker channel for that device type (e.g., a Medtronic LINQ™ marker channel). As an additional benefit, such P-wave-hunting filters facilitate arrhythmia detection, for example, by enabling model prediction for an atrial rate.

In one example, the machine learning model defined in model data 66 may be an ensemble configured to generate the positive detection for the cardiac arrhythmia based on output data from component models. Model data 66 may define an ensembling method (e.g., a board decision) for combining preliminary results from each component model of the ensemble. In one example of the ensemble, component models may be configured for respective ones of the plurality of decomposition layers where each component model may apply a set filters corresponding to the respective decomposition layer. In another example of the ensemble, component models may be configured for respective arrhythmia types where each component model comprises one or more filters configured to identify the respective arrhythmia type from the signal data.

To illustrate by way of example, the above machine learning model may be a neural network ensemble with a number of component neural networks whose output data is mathematically combined by way of some methodology. Model data 66 may define the neural network ensemble in information specifying an algorithm (e.g., logic) for generating, as output, an accurate prediction (e.g., a classification or a regression value); some examples of known ensembling methods include bootstrapping, aggregation (e.g., averaging and max voting), stacked generalization, and boosting. Model data 66 may implement the ensembling method in an ensemble neural network that is fed, as input, various data including output classes from the component neural networks. For example, model data 66 may form several neural networks into a committee in which each neural network is configured to predict one or more arrhythmia types, decomposition layers and/or the like and an ensemble (or board) network to generate a prediction for AT episodes or another specific arrhythmia type based on an evaluation of respective prediction results of the committee.

As an example of the above model, model data 66 may define a multi-layer neural network as an ensembling of different single-layer (committee) neural networks for which another single-layer (board) network determines a final prediction results by combining these neural networks in some manner. Model data 66 may define this neural network ensemble such that each neural network includes a hidden layer in which the sample of cardiac EGM data of size N is converted into a prediction result, which may be a single value, a fixed number of values, or N values. The respective predictive values of the committee networks are fed into a hidden layer of the board network for aggregation (e.g., averaging) into a final predictive value.

As demonstrated herein, filtering and filters may enhance the neural network ensemble in a number of ways by following the filter-based approach of the present disclosure. The filter-based approach encourages machine learning techniques that take advantage of filters (e.g., non-arbitrary filters and/or non-random filters) and realize improvements in terms of various performance metrics. For example, consider the model data 66 for the above neural network ensemble, model data 66 may include instructions directing processing circuitry 50 to apply the example filter to unfiltered data in the hidden layers of the committee neural networks or the hidden layer of the board neural network. One or all hidden layers may invoke the example filter to identify a decomposition layer of interest or a type of arrhythmia.

Model data 66 may prescribe one or more appropriate filters of filter(s) 68 to use in/for one or more neural network layers of a single neural network or a neural network ensemble. For example, first example filter 68 may be used in an input layer for generating input data to be fed into at least one of the component neural networks of the above neural network ensemble. As another example, processing circuitry 50 may apply first example filter 68 in an output layer of the single neural network or the neural network ensemble. As yet another example, model data 66 may direct processing circuitry 50 to apply first example filter 68 in a hidden layer of the single neural network or the neural network ensemble. In the above neural network ensemble, an output layer of each component model may invoke first example filter 68 to generate input data for the ensemble network.

Alternatively, model data 66 may define a machine learning model (e.g., a neural network) that is configured to receive filtered data as part of an (e.g., initial) input feed. As directed by model data 66 accordance with an example neural network ensemble, processing circuitry 50 may use first example filter 68 for generating a filtered data set from signal data representing cardiac activity of patient 4 and then, feeding the filtered data set to an input layer (e.g., of a component network) of the example neural network ensemble. In one example, the filtered data set may modify an amplitude or morphology of the signal data. Processing circuitry 50 may perform additional pre-processing steps to modify the filtered dataset (and further modify the signal data) in some manner prior to feeding the filtered data set to the input layer of the example neural network ensemble.

As an option, a pre-processing stage for the example neural network ensemble may include an application of first example filter 68 to signal data and/or other patient data. The pre-processing stage may include (e.g., feature extraction) for selecting first example filter 68 as an effective filter to use given a cardiac physiology of patient 4. The pre-processing stage may evaluate various patient data in addition to the signal data and therefore, further feature extraction may result in additional indicia of an arrhythmia.

Processing circuitry 50 may apply second example filter 68 to generate a filtered dataset indicative of a similarity between signal data (e.g., possibly including wavelet data or principal component data) and at least one decomposition layer of interest. Second example filter 68 may be included in one or more neural network layers such that, in accordance with the neural network, processing circuitry 50 modifies the wavelet data or principal component data to identify the at least one decomposition layer of interest, for example, as evidence of the arrhythmia and/or for input to a next neural network layer. Second example filter 68 may be configured to compare the wavelet data or the principal component data with pattern information for the expected cardiac activity of patient 4. Pattern information of the wavelet data and/or the principal component data describes one or more wavelets (e.g., R-wave, T-wave, and/or the like) and/or one or more principal components, for example, in terms of morphology, amplitude, timing, and/or the like. Second example filter 68 may generate comparison results as an example filtered dataset for which the next network layer may combine with other evidence and/or evaluate for a positive detection of the cardiac arrhythmia. Based on a totality of available evidence (e.g., in the wavelet data and/or the principal component data), processing circuitry 50 may generate output data indicative of a positive detection of the cardiac arrhythmia. In one example, processing circuitry 50 may employ a test to verify the cardiac arrhythmia and that test codifies one or more criterion for qualifying the sufficiency of the available evidence. The test may be established as a known and accurate predictor for cardiac arrhythmias.

Third example filter 68 may be included in a neural network layer (e.g., convolution layer) to correlate the signal data with a particular type of cardiac arrhythmia, for example, by determining whether pattern information (e.g., in terms of morphology, amplitude, and/or timing) of the signal data substantially matches the particular type of cardiac arrhythmia. If third example filter 68 generates a filtered dataset that converges onto a certain value or set of values, processing circuitry 50 may generate output data indicative of a positive detection of the cardiac arrhythmia.

The present disclosure introduces an ensemble neural network that is configured to generate the positive detection for the cardiac arrhythmia based on output data from at least two depth levels. Instead or in addition to a previous neural network layer, a board network is fed, as input, output data from layers of different depth levels. In some examples, a third example filter may be configured to facilitate the model prediction logic, enabling one or more layers to be omitted. In other examples, the positive detection for the cardiac arrhythmia may be based on output data from at least two depth levels without any of filter(s) 68.

Model data 66 may define one or more arrhythmia criterion for other sensor data. The machine learning model of model data 66 may apply such criterion as part of the model prediction logic. In accordance with model data 66, at least one example criterion may be directed to determining whether at least one of pulse oximeter data or accelerometer data is indicative of the cardiac arrhythmia.

In some examples, processing circuitry 50 may store one or more segments of the digitized cardiac EGM signals and then, apply filter(s) 68 to one or more portions of the stored signal data. For each portion (e.g., a decomposition layer), the stored signal data may define a morphology, a timing, and an amplitude for the cardiac activity of patient 4. Applying one or more filters 68 to the one or more portions may generate one or more filtered datasets modifying the morphology, timing, and/or amplitude for the cardiac activity of patient 4.

Each digitized cardiac EGM segment may include samples of the cardiac EGM signal spanning a configurable period of time. At least one example digitized cardiac EGM segment may be decomposed into decomposition layers of which each layer spans a length of time for which sensing circuitry 52 and/or processing circuitry 50 did indicate detection of one or more wavelets, principal components, and/or other cardiac events. In addition, a period of time before and/or after between layers may be determined. An amplitude of the cardiac EGM signal at any certain point-in-time may reflect a sum of electrical vectors in a myocardium.

Sensing circuitry 52 and/or processing circuitry 50 may be configured to decompose the cardiac EGM into waveforms (e.g., P-waves or R-waves), principal components, and any other decomposition layer of cardiac activity. As an example, the cardiac EGM may be decomposed into one or more layers of one or more cardiac depolarizations such as when the cardiac EGM amplitude crosses a sensing threshold.

Processing circuitry 50 of IMD 10, and/or processing circuitry of another device that retrieves the stored signal data from IMD 10, may analyze the above-mentioned one or more portions according to the techniques of this disclosure. The other device may be external device 12 of FIG. 1 or a server of monitoring service 6 of FIG. 1.

Processing circuitry 50 of IMD 10 may detect the cardiac arrhythmia based on a classification of the signal data in accordance with the machine learning model of model data 66. While the machine learning model may employ a plurality of filters including random filters and standardized/generic filters, the machine learning model may also invoke one or more filters 68 of which at least one filter corresponds to a feature set of the patient, wherein the feature set maps to the cardiac activity represented by at least one portion of the signal data, wherein the at least one filter 68 is applied to the at least one portion of the signal data.

As an alternative to sensing circuitry 52, processing circuitry 50 may apply an example filter 68 configured to detect a particular wavelet, principal component, and/or another cardiac event. Instead of or in addition to having sensing circuitry 52 output an indication to processing circuitry 50 in response to sensing of a particular decomposition layer such as a cardiac depolarization, processing circuitry 50 may apply filter(s) 68 to receive indicators corresponding to occurrence(s) of detected R-waves and P-waves in the respective chambers of heart. Processing circuitry 50 may use the indications of detected R-waves and P-waves for determining heart rate and detecting arrhythmias, such as tachyarrhythmias and asystole.

Processing circuitry 50 may apply an example filter 68 to one or more portions of the cardiac EGM where at least one portion may correspond to a particular decomposition layer and example filter 68 may generate an example filtered dataset indicative of each instance (e.g., location or point-in-time) of the particular decomposition layer such as R-waves or P-waves.

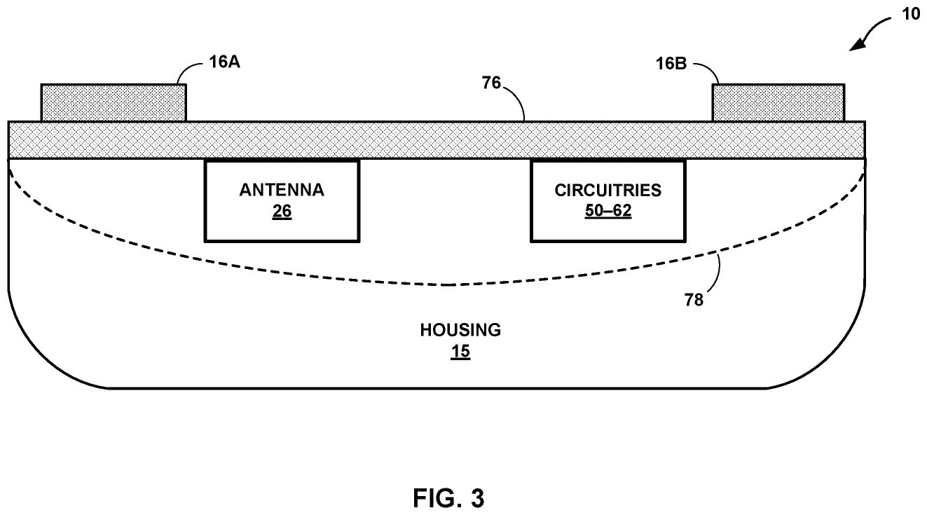

FIG. 3 is a conceptual side-view diagram illustrating an example configuration of IMD 10 of FIGS. 1 and 2. In the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. In the illustrated example, antenna 26 is formed or placed on the inner surface of cover 76, but may be formed or placed on the outer surface in some examples. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

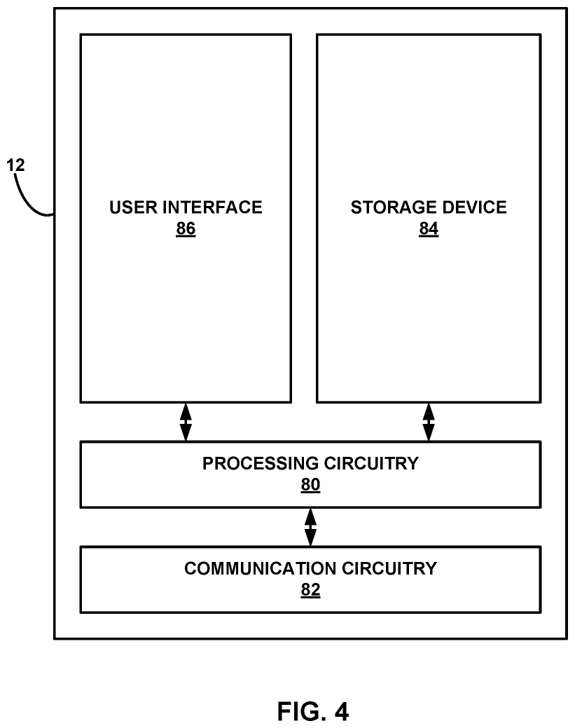

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., asystole episode data) to external device 12. In turn, external device 12 may receive the collected data from IMD 10 and store the collected data in storage device 84. Processing circuitry 80 may implement any of the techniques described herein to analyze cardiac EGMs received from IMD 10, e.g., to determine whether asystole and false asystole criteria are satisfied.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac EGMs, indications of detections of arrhythmia episodes, and indications of determinations that one or more false asystole detection criteria were satisfied. In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 5 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 5, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as asystole episode data and indications that one or more false asystole detection criteria are satisfied, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink® Network.

In some examples, server 94 may be configured to run an example computing service, such as monitoring service 6 of FIG. 1. As part of the example computing service, server 94 may maintain data in which respective feature sets are each mapped to one or more portions of signal data representing cardiac activity of one or more patients. Each feature set corresponds to one or more filters that may be configured to identify the (e.g., expected) cardiac activity of the one or more patients as represented by the one or more portions. An example filter may encode pattern information that matches or is substantially similar to an example portion of the signal data; therefore, applying the example filter to the example portion of the signal data may determine whether that example portion of the signal data represents cardiac activity that matches or is substantially similar to cardiac activity of interest (e.g., waveforms, principal components, and other cardiac events). In general, the cardiac activity of interest refers to cardiac activity that is most likely or expected to occur in the one or more patients. The example filter may be configured to identify waveforms, principal components, and other cardiac events including episodes of cardiac arrhythmias.

IMD 10 and/or external device 12 may submit, via network 92, service requests to server 94. In response to one example request having various patient data, processing circuitry 98 may extract one or more features of a feature set and identify one or more filters corresponding to the feature set.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 96, e.g., to store data retrieved from IMD 10, and processing circuitry 98. Although not illustrated in FIG. 5 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 100 may implement any of the techniques described herein to analyze cardiac EGMs received from IMD 10, e.g., to determine whether asystole and false asystole criteria are satisfied.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

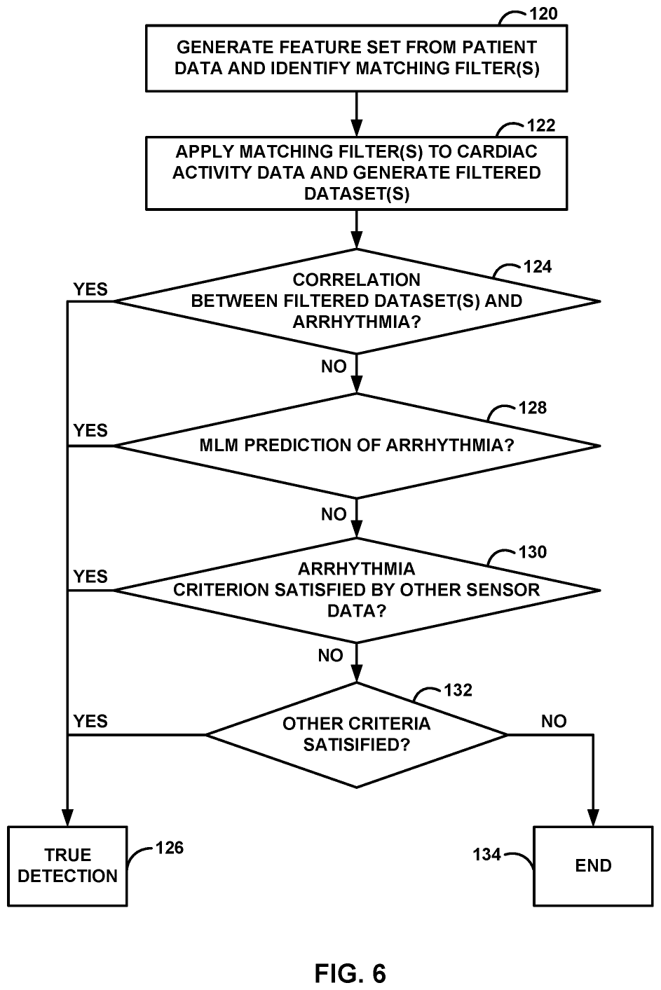

FIG. 6 is a flow diagram illustrating an example operation for a filter-based approach to detecting a cardiac arrhythmia. According to the illustrated example of FIG. 6, a medical device, such as IMD 10, is configured to determine, from signal data representing cardiac activity of a patient, whether the patient is experiencing the cardiac arrhythmia. Processing circuitry 50 of IMD 10 executes a patient-focused analysis of that signal data for indicia of a particular arrhythmia type; as described herein, the patient-focused analysis includes non-random filters which, in some instances, may be derived from historical or current cardiac activity of the patient or a corresponding patient group and therefore, may be referred to as personalized filters.

In any case, the non-random filters as described herein match a feature set of the patient and provide a number of advantages over random filters, and a medical device, such as IMD 10 of FIG. 1, having hardware/software components configured with one or more non-random filters achieves a substantial level of accuracy in distinguishing true arrhythmia episodes from false arrhythmia episodes.

The non-random matching filters may be constructed in a several manners, a non-exhaustive number of which are described in the present disclosure. Some example non-random filters derive from historical cardiac activity of the patient, encoding pattern information for one or more portions of that historical cardiac activity. The pattern information generally includes data encoding morphology, amplitude, and/or timing of a digitized signal representing a desired cardiac activity. In one example, the pattern information includes one-dimensional data representing a digitized signal of a confirmed true arrhythmia episode for the patient or at least one second patient sharing a feature set with the patient. In another example, the pattern information includes one-dimensional data representing a digitized signal of a particular decomposition layer, such as a wavelet, a principal component, or another cardiac event.

In the illustrated example of FIG. 6, processing circuitry 50 of IMD 10 generates a feature set from patient data and identifies one or more matching filters (120). IMD 10 may store mapping data (e.g., provided from monitoring server 6) where the feature set maps to the (e.g., expected) cardiac activity of at least a portion of the signal data. The patient and any second patient sharing the same feature set may have (e.g., expected) cardiac activity with same or substantially similar pattern information, and the one or more matching filters are configured to identify that cardiac activity in samples of signal data.

Processing circuitry 50 of IMD 10 executes logic to analyze one or more portions of the signal data for indica of an episode of a type of arrhythmia. A portion of the signal data may refer to a sample (e.g., of a cardiac EGM) and that sample may be of any configurable length. As part of that analysis, processing circuitry 50 of IMD 10 applies the one or more matching filters to one or more portions of signal data and generates one or more filtered datasets to identify additional and/or more accurate indicia of a type of arrhythmia (122). There are a number of ways for the one or more filtered datasets to enable IMD 10 to differentiate an episode of an arrhythmia from a non-episode.

As described in the present disclosure, an example filter may be configured to identify a particular type of cardiac arrhythmia in the signal data of the patient. In accordance with the example filter, processing circuitry 50 of IMD 10 may perform one or more vector operations on one or more portions of the signal data and generate an example filtered dataset indicative of evidence (if any) for that type of cardiac arrhythmia where, for instance, a correlation between the example filtered dataset and the particular cardiac arrhythmia may qualify as sufficient evidence (124). To determine whether there is qualifying correlation, processing circuitry 50 of IMD 10 evaluates the resulting example filtered dataset with one or more criterion for which satisfaction may indicate substantially similarity between the expected cardiac activity of the particular cardiac arrhythmia and the pattern information of the one or more portions of the signal data. One example criterion may be directed to determine whether the filtered dataset includes specific data (e.g., numerical values).

In response to determining satisfaction of the one or more criterion, processing circuitry 50 of IMD 10 confirms the correlation (YES of 124) and then, generates output data indicative of a positive detection of the particular type of arrhythmia (126). Based on determining that the example filtered dataset does not correlate with the expected cardiac activity of the particular type of arrhythmia (NO of 124), processing circuitry 50 of IMD 10 proceeds to apply a machine learning model to determine whether the model classifies the signal data as a cardiac arrhythmia (128). As described herein, IMD 10 may employ the machine learning model to distinguish an episode of the cardiac arrhythmia from non-episodes and in some instances, predict a most likely type of cardiac arrhythmia.

Based on determining that a cardiac arrhythmia is a classification of the signal data (e.g., samples of cardiac EGM) in accordance with a machine learning model (YES of 128), processing circuitry 50 of IMD 10 generates output data indicative of a positive detection of the cardiac arrhythmia (126). If that machine learning model classifies the signal data as a particular type of arrhythmia, processing circuitry 50 of IMD 10 generates output data indicative of a positive detection of the particular type of arrhythmia.

Based on determining that the machine learning model classifies the signal data as a non-episode and/or fails to classify the signal data as a cardiac arrhythmia (NO of 128), processing circuitry 50 of IMD 10 proceeds to apply arrhythmia criterion to other sensor data (130). Other than electrodes, one or more sensors (e.g., an accelerometer, a pulse oximeter, and/or the like) may provide the other sensor data and the at least one criterion is directed to determining whether at least one of pulse oximeter data or accelerometer data is indicative of the cardiac arrhythmia. Based on determining satisfaction of the arrhythmia criterion (YES of 130), processing circuitry 50 of IMD 10 generates output data indicative of a positive detection of the particular type of arrhythmia (126). If the other sensor data does not satisfy the arrhythmia criterion (NO of 130), processing circuitry 50 of IMD 10 generates output data indicative of a non-episode or, alternatively, proceeds to apply other criteria for another condition or malady.

As an option, processing circuitry 50 of IMD 10 may further analyze patient data 64 in accordance with one or more criterion. Based on a determination of at least one criterion, processing circuitry 50 of IMD 10 may generate output data indicative of a disease, a treatment side effect, a titrated treatment amount, or an implant location.

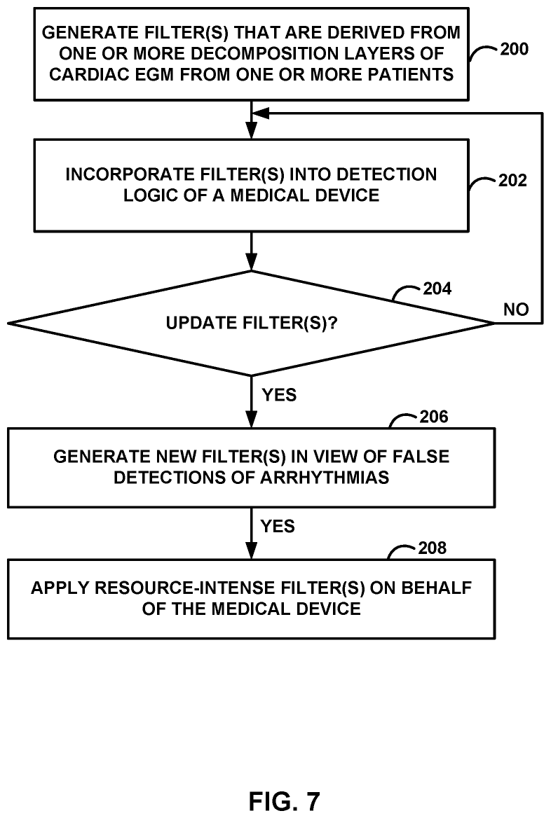

FIG. 7 is a flow diagram illustrating an example operation for generating filters that are derived from a decomposition of at least one cardiac EGM of one or more patients. According to the illustrated example of FIG. 7, processing circuitry of a computing device for monitoring service 6 (e.g., processing circuitry 98 of server 94) generates filters that are derived from one or more decomposition layers of cardiac EGM data from one or more patients (200). The cardiac EGM may be sensed by sensing circuitry 52 of IMD 10 for each patient.

Monitoring service 6 may identify a first patient based on a set of features and generate filters from the patient's cardiac EGM data. As described herein, to perform a decomposition of a cardiac EGM, monitoring service 6 may partition, into layers, individual wavelets and/or principal components of the EGM data such that each decomposition layer includes wavelet data and/or principal component data for a portion of the cardiac EGM. A filter may be derived from the wavelet data and/or principal component data of a desired decomposition layer. In this manner, the filter may be configured to identify features, such as locations, of that decomposition layer in a future cardiac EGM of that first patient. This identifying may be performed in real-time. Consider an example cardiac EGM that is decomposed (in part) into (e.g., respective) layers of R-waves and/or P-waves, where each layer defines pattern information for an R-wave or a P-wave and that pattern information may be encoded into a non-random filter. If such a filter is applied to another cardiac EGM of the same patient, a resulting filtered dataset may indicate a presence of R-wave or P-wave.

In some examples, monitoring service 6 may implement machine learning techniques to build and/or train a filter to identify a wavelet or a principal component in a given portion of the cardiac EGM. As an alternative, monitoring service 6 may build/train the filter to detect an arrhythmia from the given portion. Depending on which filter is being trained, historical cardiac EGM data provides training data including (input) features, (model) parameters, (observed) labels, and/or other data for use in personalizing/calibrating the filter for specific patient(s) or patient group(s). In addition to historical cardiac EGM data for patient 4, monitoring service 6 may store data identifying actual decompositions in the historical cardiac EGM data. For example, monitoring service 6 may partition the historical cardiac EGM data into (e.g., equal-sized/variable-sized) samples with labels indicating any detected wavelets and/or principal components and if available, whether or not a sample is indicative of an arrhythmia. A sample typically includes one or more decompositions.

As an option, monitoring service 6 may avail insight from expert reviewers to generate additional training data including observed labels for detected wavelets and/or principal components of decompositions of the historical cardiac EGM data. In this manner, if a sample of the historical cardiac EGM data has not been analyzed for a specific decomposition layer or has been analyzed but no specific decomposition layer was detected, monitoring service 6 may use an expert to classify the sample and then, use that classification as an observed label in a supervised learning technique. The expert may confirm or reject a previous detection of a wavelet and/or principal component. The expert may specify any feature(s) corresponding to (e.g., a type) of the detected wavelet and/or principal component from which a positive detection can be made. The expert-specified feature data may be transformed into filtering components in a number of known ways.

To successfully detect (at a reliable and effective rate) the same decomposition layer(s) and/or same arrhythmia type for patient 4, monitoring service 6 generates a filter, for application to unfiltered data points, as an array (e.g., one (1) dimensional vector) of n-tuples—including single values—that may be derived from the corresponding detected wavelet(s) and/or principal component(s) of the historical cardiac EGM data. Monitoring service 6 may leverage the expert's analysis to determine which values to use in the array. For instance, the array of values may be derived from the feature(s) identified by the expert such that when the array of values and the unfiltered data points are mathematically combined (e.g., via vector multiplication), the resulting filtered data may be deterministic regarding the presence of a decomposition layer of interest. The expert may identify feature data unique to patient 4's physiology for monitoring service 6 to leverage, for example, for personalizing the array of values to identify typical wavelet(s) and/or principal component(s) of patient 4. An example personalized filter for patient 4 may include an array of values matching a morphology, timing, and/or amplitude of patient 4's cardiac EGMs.

For example, if the resulting filtered data is a single value or a few values, evaluating those value(s) with one or more criterion may be deterministic as to an occurrence of particular wavelet(s) and/or principal component(s) of interest. As another example, if the resulting filtered data substantially matches a particular sequence of values, that match most likely is a positive detection of particular wavelet(s) and/or principal component(s) of interest; whereas, if the resulting filtered data is an unknown sequence, the particular wavelet(s) and/or principal component(s) of interest most likely did not occur in the corresponding sample. If possible, the filter may include data substantially matching the data points along the corresponding detected wavelet(s) and/or principal component(s) such that a comparison of those values with any given cardiac EGM sample may be deterministic. In any case, the expert may eliminate uncertainty in the training of the filter (or any model) and as a result, monitoring service 6 realizes a number of improvements.

If a sample of the historical cardiac EGM data includes a false negative or a false positive, an expert may resolve the uncertainty by determining whether or not an arrhythmia actually occurred and if so, which type occurred. The expert may specify features indicative of a true arrhythmia and whose presence, or the lack thereof, in a cardiac EGM increases a likelihood probability that the patient had a real arrhythmia. These features may be morphological, temporal, spatial, and/or the like in nature and, in some examples, may be used for classifying cardiac EGM data as a true arrhythmia of a specific type. In addition, any feature not specified by the expert may be weighted low or eliminated from the model altogether. Thus, the expert's analysis may reduce the number of possible labels as well as the number of input features, which results in fewer variables and smaller search space for the (trained) machine learning model. An overall training time for that model is reduced as a result while device performance improves (e.g., with an increased arrhythmia detection rate). The model may be approximation of a non-linear distribution and because of the above reduction, that approximation may be a linear function.

Monitoring service 6 may leverage the historical cardiac EGM data as training data for one or more filters. For an initial round of training, monitoring service 6 may generate an initial filter (e.g., a random filter), apply the initial filter to the historical cardiac EGM data to identify a particular wavelet or principal component, assess an accuracy of the initial filter, and adjust an aspect, such as pattern information, of the initial filter to be more accurate. For each subsequent round, monitoring service 6 may repeat the adjustment of the initial filter to a desired level of accuracy. Once fully trained, the resulting (e.g., personalized) filter may be calibrated for the cardiac physiology of patient 4, and monitoring service 6 may deploy the resulting filter to IMD 10.

In some examples, the first patient and at least one second patient may share the same or substantially similar pattern information between one or more decomposition layers. Monitoring service 6 may define a feature set to group together the first patient and at least one second patient based on one or more characteristics. Because fully trained filters for the first patient may also be applicable to the at least one second patient, monitoring service 6 may deploy the same filters to at least one medical device of the at least one second patient. Examples of the feature set include any combination of a patient group, a disease group, a device group, an implant location, or an implant orientation of the first patient and the at least one second patient.

In some examples, monitoring service 6 may organize samples of historical cardiac EGMs into groups where each group maps to one or more of the above example feature sets. An example group of patients having a same device may have substantially similar cardiac activity in general and/or in specific decomposition layers. By gathering samples for such an example group of patients, monitoring service 6 may generate filters that are calibrated (e.g., windowed) for historical cardiac activity.

The present disclosure introduces P-wave hunting filters, which are described in detail for FIG. 2, and these filters may be selected based on implant locations, patient body type, BMI, and/or other features. The present disclosure introduces other example filters derived from windowed cardiac EGM principal components, wavelets, and/or any other decomposition layer may include Q filter, T filter, and QT interval filters for detecting onset of QT syndromes at any length. The present disclosure also introduces example filters to correlate with cardiac EGM data for a particular type of arrhythmia, such as PVC, NSVT, SVT, PSVT, and/or the like.

As described herein, monitoring service 6 may run an example computing service for IMD 10 and other medical devices. In response to a service request, monitoring service 6 may generate, and then return, one or more personalized filters for patient 4. In turn, each medical device incorporates the filters into detection logic (202) and in some examples, processing circuitry 50 of IMD 10 may apply the incorporated filters to improve upon device performance. For example, as discussed in greater detail with respect to FIGS. 1 and 6, processing circuitry 50 may apply a filter to generate a filtered dataset indicative of each cardiac depolarization, e.g., R-wave, within the cardiac EGM.

As another service request, IMD 10 may submit recorded cardiac EGM data to monitoring service 6, which determines whether to update any of the generated filters (204). If, for instance, the cardiac EGM data corresponds to one or more false detections of arrhythmias, monitoring service 6 may decide to update the filters (YES of 204) by generating new filters in view of the false detections (206). Monitoring service 6 may generate the new filters based on pattern information of the subsequent cardiac EGM data. However, if the subsequent cardiac EGM data includes substantially the same pattern information, monitoring service 6 may decide not to update the filter (NO of 204) and return to incorporating filter(s) into detection logic of IMD 10 (202).

In some examples, after observing a pre-defined quantity of false detections and other errors, IMD 10 may automatically submit a request for monitoring service 6 to update the current filters for IMD 10. Over a number of iterations, monitoring service 6 may modify a given filter to model more precisely the target aspect of the cardiac physiology of patient 4. In this manner, existing filters may undergo fine-tuning in view of additional training data, and if monitoring service 6 has updated the current filters of IMD 10, the techniques described herein may task any number of technologies to make the updated filters available (e.g., via a wireless connection, such as an Internet connection). After receiving (e.g., downloading) and then, incorporating (e.g., programming) the updated filters into the detection logic, IMD 10 may proceed to apply those updated filters in place of the current filters. IMD 10 may realize improved results when evaluating the subsequent cardiac EGMs, and patient 4 benefits from any increased accuracy resulting from the update.

In some examples, monitoring service 6 may have substantially more resources than IMD 10 and thus, may be configured to run resource-intense filters. For at least this reason, monitoring service 6 may support arrhythmia detection at IMD 10 by enabling access to these filters. For example, monitoring service 6 may run a cloud computing service on a server that, when requested by IMD 10, is configured to invoke a specific resource-intense filter and generate filtered data to be returned to IMD 10. IMD 10 may request the specific resource-intense filter, or, as an alternative, have monitoring service 6 select an appropriate filter. Through an interface, IMD 10 may submit unfiltered data in an example service request for the server running monitoring service 6 to handle. As directed in the request, the server may receive the unfiltered data and in turn, apply the appropriate resource-intense filter on behalf of IMD 10 (208). Depending on which filter is applied, monitoring service 6 may return any filtered data to the IMD 10.

In response to a false AF detection, IMD 10 may request that monitoring service 6 apply filters configured to identify one or more other types of arrhythmias, such as Tachycardia or PVC. In some instances, Tachycardia or PVC causes the false AF detection and the filters applied on the server increase specificity over filters applied on IMD 10.

The order and flow of the operations illustrated in FIG. 6 and FIG. 7 are one examples. In other examples according to this disclosure, more or fewer operations may be considered in a different order, or satisfaction of different numbers or combinations of operations may be required for an evaluation of cardiac EGM data. Further, in some examples, processing circuitry may perform or not perform the method of FIG. 6 or the method of FIG. 7, or any of the techniques described herein, as directed by a user, e.g., via external device 12 or computing devices 100. For example, a patient, clinician, or other user may turn on or off functionality for identifying true or false arrhythmias remotely (e.g., using Wi-Fi or cellular services) or locally (e.g., using an application provided on a patient's cellular phone or using a medical device programmer).

Additionally, although described in the context of an example in which IMD 10, and processing circuitry 50 of IMD 10, perform each of the portions of the example operation, the example operation of FIG. 6, as well as the example operations described herein with respect to FIG. 7, may be performed by any processing circuitry of any one or more devices of a medical system, e.g., any combination of one or more of processing circuitry 50 of IMD 10, processing circuitry 80 of external device 12, processing circuitry 98 of server 94, or processing circuitry of computing devices 100.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Example 1: A medical system includes one or more sensors configured to sense cardiac activity of a patient; sensing circuitry configured to generate signal data to represent the cardiac activity of the patient; and processing circuitry configured to: detect a cardiac arrhythmia for the patient based on a classification of the cardiac activity in accordance with a machine learning model, wherein the machine learning model comprises at least one filter corresponding to a feature set of the patient and configured for application to at least one portion of the signal data; and generate output data indicative of a positive detection of the cardiac arrhythmia.

Example 2: The medical system of example 1, wherein the at least one filter is derived from at least one of cardiac EGM data of the patient or second cardiac EGM data of at least one second patient, wherein the at least one second patient corresponds to the feature set of the patient.

Example 3: The medical system of example 2, wherein the at least one filter comprises pattern information of at least one decomposition layer of the second cardiac EGM data.

Example 4: The medical system of any of examples 1 through 3, wherein the feature set comprises at least one of a patient group, a disease group, a device group, an implant location, or an implant orientation of the patient.

Example 5: The medical system of any of examples 1 through 4, wherein to detect a cardiac arrhythmia, the processing circuitry is configured to identify at least one decomposition layer in the signal data based on an application of the at least one filter.

Example 6: The medical system of any of examples 1 through 5, wherein the processing circuitry is further configured to update the at least one filter based on at least one decomposition layer of the signal data.

Example 7: The medical system of any of examples 1 through 6, wherein the processing circuitry is further configured to modify wavelet data or principal component data to identify at least one decomposition layer of the signal data.

Example 8: The medical system of any of examples 1 through 7, wherein the machine learning model comprises, for each of a plurality of decomposition layers, a set of one or more filters derived from data associated with that respective one of the plurality of decomposition layers.

Example 9: The medical system of any of examples 1 through 8, wherein the machine learning model further comprises an ensemble configured to generate the positive detection for the cardiac arrhythmia based on output data from component models.

Example 10: The medical system of example 9, wherein the ensemble further comprises component models for respective ones of a plurality of decomposition layers, wherein each component model comprises a set of filters corresponding to the respective decomposition layer.

Example 11: The medical system of any of examples 9 and 10, wherein the ensemble further comprises component models for respective arrhythmia types, wherein each component model comprises one or more filters configured to identify the respective arrhythmia type from the signal data.

Example 12: The medical system of any of examples 1 through 11, wherein the machine learning model comprises at least one criterion directed to determining whether at least one of pulse oximeter data or accelerometer data is indicative of the cardiac arrhythmia.

Example 13: The medical system of any of examples 1 through 12, wherein the processing circuitry is further configured to generate, based on a determination of at least one criterion, output data indicative of a disease, a treatment side effect, a titrated treatment amount, or an implant location.

Example 14: The medical system of any of examples 1 through 13, wherein to detect a cardiac arrhythmia, the processing circuitry is configured to modify at least one of an amplitude, a timing, or a morphology of principal component data or wavelet data of the at least a portion of the signal data.

Example 15: The medical system of any of examples 1 through 14, wherein the machine learning model comprises an ensemble configured to generate the positive detection for the cardiac arrhythmia based on output data from at least two depth levels of the machine learning model.

Example 16: The medical system of any of examples 1 through 15, wherein the machine learning model comprises an ensemble configured to generate the positive detection for the cardiac arrhythmia based on filtered datasets of the signal data.

Example 17: A method includes generating, by sensing circuitry coupled to one or more sensors, signal data to represent cardiac activity of the patient; detecting, by processing circuitry, a cardiac arrhythmia for the patient based on a classification of the signal data in accordance with a machine learning model, wherein the machine learning model comprises at least one filter that is configured for application to at least one portion of the signal data and maps to a feature set indicative of a cardiac physiology of the patient; and generating, by the processing circuitry, output data indicative of a positive detection of the cardiac arrhythmia.

Example 18: The method of example 17, wherein the at least one filter comprises pattern information of at least one decomposition layer of the second cardiac EGM data.

Example 19: The method of any of examples 17 and 18, wherein the feature set comprises at least one of a patient group, a disease group, a device group, an implant location, or an implant orientation of the patient.

Example 20: A non-transitory computer-readable storage medium includes generate patient data corresponding to at least one physiological parameter of the patient, wherein the patient data comprises signal data to represent electronic activity of a heart of the patient, wherein the medical system comprises one or more sensors configured to sense the electrical activity and sensing circuitry, coupled to the one or more sensors, configured to generate the signal data; detect a cardiac arrhythmia for the patient based on a classification of the patient data in accordance with a machine learning model configured for the at least one physiological parameter of the patient, wherein the machine learning model comprises a plurality of filters of which at least one filter is applied, based on the patient data, to at least one portion of the signal data; and generate output data indicative of a positive detection of the cardiac arrhythmia.

What is claimed is:

1. A medical system comprising:
one or more sensors configured to sense cardiac activity of a patient;
sensing circuitry configured to generate signal data to represent the cardiac activity of the patient; and
processing circuitry configured to:
detect a cardiac arrhythmia for the patient based on a classification of the cardiac activity in accordance with a machine learning model, wherein the machine learning model comprises at least one filter corresponding to a feature set of the patient and configured for application to at least one portion of the signal data; and
generate output data indicative of a positive detection of the cardiac arrhythmia.

2. The medical system of claim 1, wherein the at least one filter is derived from at least one of cardiac EGM data of the patient or second cardiac EGM data of at least one second patient, wherein the at least one second patient corresponds to the feature set of the patient.

3. The medical system of claim 2, wherein the at least one filter comprises pattern information of at least one decomposition layer of the second cardiac EGM data.

4. The medical system of claim 1, wherein the feature set comprises at least one of a patient group, a disease group, a device group, an implant location, or an implant orientation of the patient.

5. The medical system of claim 1, wherein to detect a cardiac arrhythmia, the processing circuitry is configured to identify at least one decomposition layer in the signal data based on an application of the at least one filter.

6. The medical system of claim 1, wherein the processing circuitry is further configured to update the at least one filter based on at least one decomposition layer of the signal data.

7. The medical system of claim 1, wherein the processing circuitry is further configured to modify wavelet data or principal component data to identify at least one decomposition layer of the signal data.

8. The medical system of claim 1, wherein the machine learning model comprises, for each of a plurality of decomposition layers, a set of one or more filters derived from data associated with that respective one of the plurality of decomposition layers.

9. The medical system of claim 1, wherein the machine learning model further comprises an ensemble configured to generate the positive detection for the cardiac arrhythmia based on output data from component models.

10. The medical system of claim 9, wherein the ensemble further comprises component models for respective ones of a plurality of decomposition layers, wherein each component model comprises a set of filters corresponding to the respective decomposition layer.

11. The medical system of claim 9, wherein the ensemble further comprises component models for respective arrhythmia types, wherein each component model comprises one or more filters configured to identify the respective arrhythmia type from the signal data.

12. The medical system of claim 1, wherein the machine learning model comprises at least one criterion directed to determining whether at least one of pulse oximeter data or accelerometer data is indicative of the cardiac arrhythmia.

13. The medical system of claim 1, wherein the processing circuitry is further configured to generate, based on a determination of at least one criterion, output data indicative of a disease, a treatment side effect, a titrated treatment amount, or an implant location.

14. The medical system of claim 1, wherein to detect a cardiac arrhythmia, the processing circuitry is configured to modify at least one of an amplitude, a timing, or a morphology of principal component data or wavelet data of the at least a portion of the signal data.

15. The medical system of claim 1, wherein the machine learning model comprises an ensemble configured to generate the positive detection for the cardiac arrhythmia based on output data from at least two depth levels of the machine learning model.

16. The medical system of claim 1, wherein the machine learning model comprises an ensemble configured to generate the positive detection for the cardiac arrhythmia based on filtered datasets of the signal data.

17. A method comprising:
generating, by sensing circuitry coupled to one or more sensors, signal data to represent cardiac activity of the patient;
detecting, by processing circuitry, a cardiac arrhythmia for the patient based on a classification of the signal data in accordance with a machine learning model, wherein the machine learning model comprises at least one filter that is configured for application to at least one portion of the signal data and maps to a feature set indicative of a cardiac physiology of the patient; and
generating, by the processing circuitry, output data indicative of a positive detection of the cardiac arrhythmia.

18. The method of claim 17, wherein the at least one filter comprises pattern information of at least one decomposition layer of the second cardiac EGM data.

19. The method of claim 17, wherein the feature set comprises at least one of a patient group, a disease group, a device group, an implant location, or an implant orientation of the patient.

20. A non-transitory computer-readable storage medium comprising program instructions that, when executed by processing circuitry of a medical system, cause the processing circuitry to:
generate patient data corresponding to at least one physiological parameter of the patient, wherein the patient data comprises signal data to represent electronic activity of a heart of the patient, wherein the medical system comprises one or more sensors configured to sense the electrical activity and sensing circuitry, coupled to the one or more sensors, configured to generate the signal data;
detect a cardiac arrhythmia for the patient based on a classification of the patient data in accordance with a machine learning model configured for the at least one physiological parameter of the patient, wherein the machine learning model comprises a plurality of filters of which at least one filter is applied, based on the patient data, to at least one portion of the signal data; and
generate output data indicative of a positive detection of the cardiac arrhythmia.
